(19) United States
(12) Patent Application Publication
(10) Pub. No.: US 2005/0065485 A1
(43) Pub. Date: Mar. 24, 2005

(54) Ostomy appliance

(76) Inventors: Birthe Vestbo Andersen, Espergaerde (DK); Hans Olsen, Hoersholm (DK)

Correspondence Address: JACOBSON HOLMAN PLLC, 400 SEVENTH STREET N.W., SUITE 600, WASHINGTON, DC, 20004, US

(21) Appl. No.: 10/490,426

(22) PCT Filed: Sep. 23, 2002
(86) PCT No.: PCT/DK02/00622

(30) Foreign Application Priority Data
Sep. 24, 2001 (DK) PA 2001 01390

(51) Int. Cl. A61F 5/44

(52) U.S. Cl. 604/332

(57) ABSTRACT

An ostomy appliance comprising a front wall and a rear wall of flexible material forming a bag, the rear wall having an inlet opening into the bag by which waste material can enter the bag and one of the walls optionally has one or more vents through which gas may escape from the bag and having a filter covering said vent wherein gas inlet and outlet openings are provided in communication with a filter body, wherein at least an area of the wall immediate opposite the inlet opening is provided with a coating reducing the friction and the cohesion between the walls wherein also a part of the inner surface of the wall comprising the inlet opening is provided with a coating reducing the surface friction coefficient. Thus the adhesion between the walls is reduced and they will then be easier to separate for the material entering a fresh bag and gravitation will then force the material against the bottom of the bag and reduce the risk of blocking of the entrance of the bag and or a filter.

### BACKGROUND OF THE INVENTION

1. Field of the Invention

The present invention relates to an ostomy appliance comprising a deodorising filter, in particular ostomy bags, and to a filter device.

In connection with surgery for a number of diseases in the gastro-intestinal tract a consequence is, in many cases, that the patient is left with a stoma such as a colostomy, an ileostomy or an urostomy in the abdominal wall for the discharge of the effluents or waste products of the body, which are conveyed through the colon, the ileum or the ureter. The discharge of visceral contents including intestinal gases cannot be regulated at will, and for that purpose the opening may be closed with a closure means, e.g. a tampon or a magnetic closure, or the patient will have to rely on an appliance to collect the material emerging from such opening in the form of a receiving bag which is later emptied and/or discarded at suitable times.

Ostomy appliances are well known. Such appliances may be two-piece or one-piece appliances. In both types of appliances, an adhesive wafer is attached to the wearer's skin. In case of a one-piece appliance, a receiving member or bag is attached to the adhesive wafer. In case of a two-piece appliance, the adhesive wafer forms part of a body side member and a receiving bag is attached releasably to the body side ostomy member for receiving exudates from the stoma.

When using one-piece appliances, the whole appliance, including the adhesive skin barrier securing the appliance to the skin is normally removed and replaced by a fresh appliance. When using two-piece appliances, the body side member is left in place up to several days, and only the receiving bag attached to the body side member is replaced.

The discharge of flatus, measured in volume, may exceed the discharge of solid and liquid faecal matter by many hundred percent and therefore there is usually the need for the continuous or frequent venting of the intestine or the collecting bag. Normally the outflowing flatus is deodorised with a suitable filter. Commonly the active filter is powdered active carbon, which absorbs H₂S being the principal component of the smell of flatus.

2. Description of the Related Art

Various constructions of filters for ostomy appliances are known. In the state of the art, the filters are designed so as to obtain a high security of deodorisation of the flatus by securing that there is no by-pass by which the flatus may circumvent the filter, and some measures have been discussed with respect to obtaining a better security against blocking of the inlet of the filter by solid discharged visceral content. When the inlet opening of the filter is blocked, the gas discharged into the ostomy appliance cause ballooning of the bag which is highly undesirable for several reasons. Ballooning will be embarrassing for the user as the bag will bulge and there is an increased risk of leakage which is unacceptable for the user.

Several solutions for alleviating this risk of blocking of the filter have been proposed. These solutions have been concentrated on the measures physically preventing solid visceral content discharged from a stoma from reaching the inlet opening(s) of the filter and thus from blocking the same. Such solutions are, however, not always fully satisfactory for overcoming the above problems. Due to adhesion between the walls of a fresh receiving bag there is still a considerable risk that visceral contents being discharged from a stoma will not immediately be able to separate the walls and “fall” into the bottom of the receiving bag but will rather stay and block the entrance to the bag or even pass upwards against the inlet opening of a filter of the bag which will again increase the risk of blocking of the filter resulting in ballooning and even leakage. The same also applies for bags without a filter in which case a blocking of the bag immediately around the inlet opening may reduce the part of the bag utilised and consequently reduce the service time of the bag and also increase the risk of soiling or contamination the distal surface and the coupling area of a base plate. Thus, there is still a need for measures alleviating these problems.

U.S. Pat. No. 4,518,388 discloses an ostomy bag having an insert being placed inside the bag at the wall opposite the wall of the bag comprising the inlet, said insert being produced from a material having a low surface friction such as silicone.

### SUMMARY OF THE INVENTION

The present invention relates to an ostomy appliance comprising a front wall and a rear wall of flexible material forming a bag, the rear wall having an inlet opening into the bag by which waste material can enter the bag and one of the walls optionally has one or more vents through which gas may escape from the bag and having a filter covering said vent wherein gas inlet and outlet openings are provided in communication with a filter body.

### BRIEF DESCRIPTION OF THE DRAWINGS

The invention is disclosed more in detail with reference to the drawings in which

FIG. 1 shows a sectional view of an embodiment of an ostomy receiving bag of the invention, and

FIG. 2 shows a sectional view of another embodiment of an ostomy receiving bag of the invention.

### DETAILED DESCRIPTION OF THE PRESENT INVENTION

The present invention relates to an ostomy appliance comprising a front wall and a rear wall of flexible material forming a bag, the rear wall having an inlet opening into the bag by which waste material can enter the bag and one of the walls optionally has one or more vents through which gas may escape from the bag and having a filter covering said vent, wherein at least an area of the inner surface of the wall immediate opposite the inlet opening is provided with a coating reducing the surface friction coefficient and the adhesion between the walls. It is also considered a part of the invention to provide at least a part of the inner surface of the wall comprising the inlet opening is with a coating reducing the surface friction coefficient.

In a bag according to the invention the coating will reduce the friction of the waste materials entering the bag against the opposite wall and thus the tendency of adhering to the wall and furthermore it will facilitate the separation of the walls by reducing the adhesion and friction between the walls which will then be easier to separate for the material entering a fresh bag in which the walls often adhere to each other, and gravitation will then force the material against the bottom of the bag and reduce the risk of blocking of the bag and of a filter inlet opening. Thus, the present invention presents a solution to the above problems.

In order to secure an easier separation of the walls of the receiving bag it is preferred that the area of the wall immediate opposite the inlet opening which has a larger extension than the diameter of the inlet opening of the bag. The area of the wall being provided with a coating reducing the surface friction coefficient may optionally be much larger than the diameter of the opening and may even cover the whole surface.

In accordance with a preferred embodiment of the invention, also a part of the inner surface of the wall comprising the inlet opening is provided with a coating reducing the surface friction coefficient. Such area is suitably in the form of a ring-shaped area immediately neighbouring the rim of the inlet opening and may also cover a larger part or all of the inner surface of this wall.

A bag according to this preferred embodiment of the invention differs from the bags known from U.S. Pat. No. 4,518,388 in that a part of both walls are covered with such a coating. The effect of this is that it is rendered more easy for the material entering a fresh appliance to separate the walls due to reduced ahherence and furthermore, there is no risk either that the material would adhere to the wall comprising the inlet opening and cause a blocking.

It is preferred when the coating is in the form of a coating showing a lower coefficient of friction when wet than when dry as the environment in an ostomy receiving bag is very humid. Thus, it has been found to be suitable when the coating is in the form of adhesive having a surface showing hydrophilic properties. Such adhesive may be any adhesive known per se adhering safely to the wall of the bag and being stable in the aggressive environment of the visceral contents and not causing any adverse effect when in contact with human mucosal or skin surfaces such as the stoma or its environments. The adhesive may be an adhesive showing hydrophilic properties in itself or it may alternatively be provided with a surface showing hydrophilic properties, e.g. by adhering hydrophilic material to the surface. Suitable adhesives are e.g. the adhesives disclosed in U.S. Pat. No. 4,367,732 or in European published Application Nos. EP 980268 or EP 985006.

The hydrophilic material may e.g. be in the form of a hydrophilic powder such as CMC powder covering the surface.

It is especially preferred if the coating is in the form of a coating of a hydrophilic polymer such as a coating conventionally being used for coating e.g. catheters for intermittent catherisation. The properties of such coatings are very similar to and considered safe for use in contact with mucosal surfaces and may suitably be those disclosed in U.S. Pat. No. 5,001,009 or European Application No. EP 991701 which will readily adhere to a bag wall made from the materials conventionally used in the art.

An adhesive and a hydrophilic powder may be applied in a manner known per se in-line in a station during the manufacture of the ostomy bag.

A hydrophilic coating may applied in a manner known per se e.g. by a coating or printing process such as serigraphy or rotogravure and the coating may, if desired, be cured using e.g. UV light.

However, some of these processes may involve processing steps involving influence by temperature and/or solvents which might adversely affect the properties of the material for the walls of the bag.

Thus, it may be suitable to prepare a disc being able to withstand such influences and then to affix such a disc to the wall of the bag in a separate step.

It is also considered an embodiment of the invention to provide a separate disc of a suitable material provided with a surface having the desired surface properties which disc may then be secured to the wall of the bag using conventional techniques such as welding or gluing in a suitable step of the production process.

Using a separate disc may also contribute in imparting some stiffness to the wall of an ostomy receiving bag immediately opposite the inlet hole which may also facilitate the separation of the walls of a fresh bag and furthermore reduce the risk of a collapsing of the bag through folding of the upper part thereof constricting the access to the upper part and to a vent which might lead to ballooning and reduced utilisation of the space of the bag.

Such a disc may e.g. be made from a sheet of polyurethane material being provided with a desired surface coating on one surface.

The coating may also be a hydrophobic coating such as a surface covered with a material such as a silicone polymer or a tetrafluoroethylene polymer providing a very smooth surface with low tendency of adhering and friction. Such a material may e.g. be applied using a plasma treatment.

The ostomy receiving bag according to the invention may be adapted for use together with an ostomy body side member (2-piece appliance) wherein the receiving bag is provided with coupling means for releasable securing to matching coupling means placed on the ostomy body side member and wherein the inlet opening is adapted for alignment with a hole of the ostomy body side member for receiving a stoma.

The ostomy receiving bag according to the invention may, as an alternative, be adapted for use directly (1-piece appliance) in which case the bag is provided with an adhesive wafer for securing the receiving bag to the user's skin, said bag and wafer having an inlet opening for receiving a stoma.

As indicated above, an ostomy receiving bag of the invention may be provided with a vent. Such bags are normally closed bags.

In accordance with a preferred embodiment an ostomy receiving bag of the invention is in the form of an open bag in which one or preferably both surfaces of the parts of the walls forming the closable outlet are provided with a coating reducing the surface friction coefficient. This is especially suitable as the emptying of the bag and also the cleaning of the outlet is then facilitated.

The receiving bag itself comprising front and rear walls sealed together along the rim and provided with an inlet opening may be made in analogy with and from materials conventionally used for the preparation of ostomy appliances.

Such materials are suitably films composed of any suitable material which is heat sealable and sufficiently impervious for unpleasant odours such as polyolefin films or combinations of such films, e.g. polyethylene or a coextrudate of polyethylene and polyvinylidene chloride.

An ostomy body side member for use together with an ostomy receiving bag according to the invention may be produced from standard materials normally used for preparation of disposable ostomy and wound and incontinence devices.

Thus, the adhesive wafer for a body side member or of a 1-piece ostomy appliance bag according to the invention may be made from a medical grade barrier adhesives known in the such as the formulation being disclosed, for example in U.S. Pat. Nos. 4,367,732, 5,051,259 or 5,714,225. For a 2-piece ostomy appliance according to the invention the body side member and the receiving bag are provided with matching coupling means.

The coupling means for use in connection with the present invention may be any suitable coupling means known per se for coupling of ostomy base plates to ostomy collecting bags, e.g. a mechanical coupling such as matching coupling rings such as the coupling rings disclosed in WO 91/01118 and WO 91/01119 or WO 94/18919 or matching flanges for adhesive connection of the type disclosed in U.S. Pat. No. 5,800,415.

### DESCRIPTION OF THE PREFERRED EMBODIMENTS

The invention is now explained more in detail with reference to the drawings showing preferred embodiments of the invention.

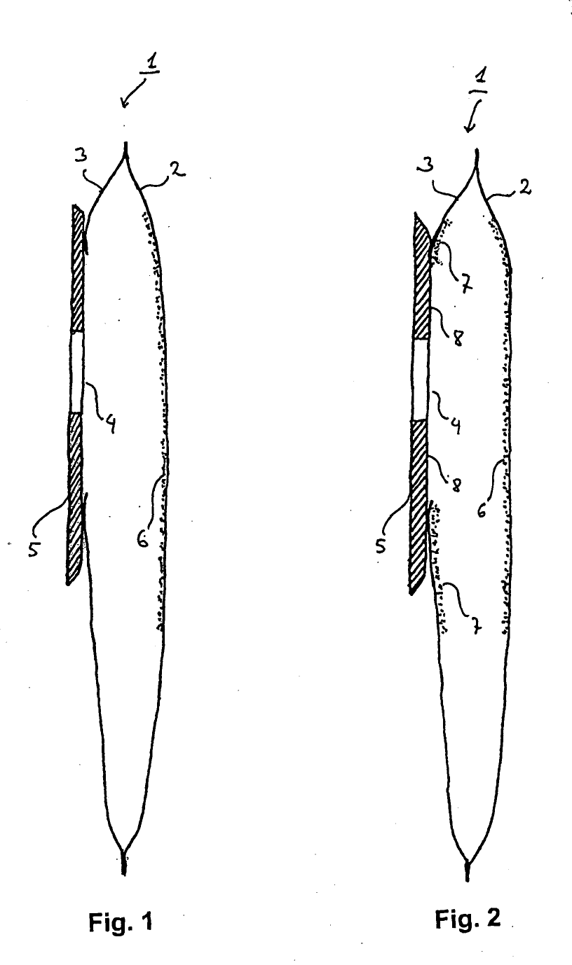

Reference is made to FIG. 1 of the drawings showing a sectional view of an embodiment of a closed ostomy receiving bag of the invention.

The bag generally designated 1 comprises a front wall 2 and a rear wall 3 of flexible material forming a bag, the rear wall having an inlet opening 4 into the bag by which waste material can enter the bag. The embodiment shown is a one-piece appliance having an adhesive wafer 5 for adhering to the user's skin. As appears, the area 6 of the front wall 2 immediate opposite the inlet opening 4 is provided with a coating reducing the surface friction coefficient which coating has a larger extension than the diameter of the inlet opening of the bag.

FIG. 2 shows a sectional view of another embodiment of a closed ostomy receiving bag of the invention in which corresponding elements have corresponding referal numbers. In this embodiment also an area 7 of the rear wall 3 is provided with a coating reducing the surface friction coefficient. In this embodiment the area is surrounding the inlet opening but not covering the surface 8 immediately neighbouring the rim of the inlet opening 4.

1. An ostomy appliance comprising a front wall and a rear wall of flexible material forming a bag, the rear wall having an inlet opening into the bag by which waste material can enter the bag and one of the walls having one or more vents through which gas may escape from the bag and having a filter covering said vent, wherein at least an area (6) of the wall immediate opposite the inlet opening (4) is provided with a coating reducing the surface friction coefficient and the adhesion between the walls, characterised in that a part of the inner surface of the wall comprising the inlet opening is provided with a coating reducing the surface friction coefficient.

2. An ostomy appliance as claimed in claim 1, wherein the area has a larger extension than the diameter of the inlet opening of the bag.

3. An ostomy appliance as claimed in claim 1, wherein the coating is in the form of a coating showing a lower coefficient of friction when wet than when dry.

4. An ostomy appliance as claimed in claim 1, wherein the coating is in the form of adhesive provided with a surface showing hydrophilic properties.

5. An ostomy appliance as claimed in claim 1, wherein the coating is in the form of a coating of a hydrophilic polymer.

6. An ostomy appliance as claimed in claim 1 wherein the bag is in the form of an open bag in which one or preferably both surfaces of the parts of the walls forming the closable outlet are provided with a coating reducing the surface friction coefficient.

7. An ostomy appliance as claimed in claim 1 wherein a separate disk of a suitable material provided with the coating is secured to the wall of the bag immediately opposite the inlet hole.

8. An ostomy appliance comprising a front wall and a rear wall of flexible material forming a bag, the rear wall having an inlet opening into the bag by which waste material can enter the bag and one of the walls having one or more vents through which gas may escape from the bag and having a filter covering said vent, wherein at least an area (6) of the wall immediate opposite the inlet opening (4) is provided with a coating reducing the surface friction coefficient and the adhesion between the walls, characterised in that a separate disk of a suitable material provided with the coating is secured to the wall of the bag immediately opposite the inlet hole.
